(19) United States
(12) Patent Application Publication
(10) Pub. No.: US 2005/0059892 A1
(43) Pub. Date: Mar. 17, 2005

(54) Method and system for lateral gain control in an ultrasound imaging system

(76) Inventors: Elizabeth Dubois, Portsmouth, NH (US); David J. Kuzara, Chelmsford, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/941,323

(22) Filed: Sep. 15, 2004

Related U.S. Application Data
(60) Provisional application No. 60/503,847, filed on Sep. 17, 2003.

(51) Int. Cl. A61B 8/00

(52) U.S. Cl. 600/443

(57) ABSTRACT

An ultrasound imaging system lateral gain control circuit operates in conjunction with an ultrasound imaging system for generating an ultrasound image of an ultrasound analysis object using an ultrasound sensing circuit for sensing an ultrasound signal returning from an ultrasound analysis object to an ultrasound transducer. The ultrasound image is defined by a first region and a second region. The first region and second region together form the complete ultrasound image. The first region associates with a region proximate to the ultrasound transducer, while the second region associates with a region distal from the ultrasound transducer. The lateral gain control circuit controls the two-dimensional ultrasound image brightness between the two regions by controlling the ultrasound signal amplification by separately controlling the first region signal amplification and the second region signal amplification.

### CROSS REFERENCE TO RELATED CASES

Applicants claim the benefit of Provisional Application Ser. No. 60/503,847, filed Sep. 17, 2003.

The present invention relates to ultrasound systems and their methods of operation and, in particular, to an ultrasound imaging system lateral gain control circuit and method of operation for controlling the lateral gain in an ultrasound image.

Diagnostic ultrasound imaging systems transmit sound energy into the human body and receive signals reflecting from tissue and organs such as the heart, liver, and kidney. In general, these systems convert echoes received from objects on the plurality of paths into electrical signals from which they generate and display an ultrasound image in a two-dimensional format known as color flow imaging or color velocity imaging. The amplitudes of reflected components for structures such as the heart or vessel walls have lower absolute velocities and are 20 dB to 40 dB (10-100 times) larger than reflected components due to blood cells.

One important aspect of ultrasound imaging system includes operator adjustable gain control for controlling the gain of an ultrasonic scanning system as a function of the scanning angle of a received beam. Existing ultrasonic scanning systems employ several techniques for controlling transmit or receive channel gains. At least some of these gains are operator adjustable to help present the desired image on the display screen. For example, the SONOS line of ultrasound systems, which is manufactured by PHILIPS MEDICAL SYSTEMS, incorporates an operator adjustable transmit gain control mechanism. Varying this control affects the intensity of the entire signal that the ultrasound image monitor displays. In addition, the time gain compensation (known as “TGC”) function allows an operator to adjust the receive channel gain in discrete steps as a function of distance from the origin of the beam. TGC control permits the adjustment of gain to compensate for the attenuation occurring as the ultrasound signal as it propagates through the scanned medium.

At times, it also may be desirable to modify the gain, and hence the intensity of the displayed signal, as a function of the scan angle. This may enable the clinician to better analyze a particular region of a subject's anatomy, such as the motion of a wall of the heart. Such a gain adjustment, oriented along scan lines or within a range of scan angles, also may be desirable if the ultrasound signal is decreased in a particular region by shadowing, caused by anatomical structures such as the ribs or lungs of the subject being scanned, or where tissue fiber orientation of the subject results in displayed signals having unacceptable brightness. Finally, such an adjustment may be desirable in a system utilizing techniques for automatically detecting and highlighting such border regions as the interface between the wall and a chamber of the subject's heart. Absent such an adjustment, in regions where shadowing appears, there may not be sufficient signal for such automatic detection circuits to effectively function. U.S. Pat. No. 5,195,521, which is assigned to the assignee of the present application, describes such the operation of such a detection circuit.

Currently only cardiac ultrasound systems use LGC controls that allow the user to control the relative gain applied to various lateral sections of the image. LGC control typically reduces the echo intensity of the cardiac walls to get a more uniform image. While the LGC controls could be highly useful for non-cardiac applications in allowing the clinician to optimize image presentation, no current specialty or general imaging system provides LGC. For example, refraction from the baby's head or stomach sidewalls can make the image too dim at the sides to be viewed easily. In abdominal studies, a rib may cause shadowing and make one side of the image too dim. Similarly, a gas pocket may make one side of the image too bright. Large disparities in image brightness across an image can make it difficult or impossible to get accurate measurements of anatomy.

In prior gain adjustment systems oriented along scan lines or within a range of scan angles, the individual controls have been either unduly complex or adapted from the earlier TGC type of controls. For example, the SONOS line of gain adjustment controls use an 8-slider system called lateral gain control (LGC) to adjust different aspects of scan line or scan angles. Unfortunately, this creates significant problems relating to the complexity of understanding how to adjust these controls, the effect of different controls, and the general expense of the hardware and circuitry in manufacturing these controls. The 8-slider system presents to the clinician many choices and may ultimately result in inferior performance, due to these controls being improperly positioned. Accordingly, a need exists for a simpler way to control the image display gain for scan line and scan angle images.

In accordance with the present invention, a method and system for controlling the lateral gain of an ultrasound image is provided that substantially eliminates or reduces the disadvantages and problems associated with prior ultrasound image system display gain control methods and systems.

According to one aspect of the present invention, the present invention provides a LGC circuit and method of operation for laterally controlling the gain of an ultrasound image generated by an ultrasound imaging system. The present invention generates an ultrasound image of an ultrasound analysis object using an ultrasound sensing circuit for sensing an ultrasound signal returning from an ultrasound analysis object to an ultrasound transducer. The ultrasound image includes a first region and a second region. The first region and the second region together form the ultrasound image with the first region associating with a region of the ultrasound analysis object proximate to the ultrasound transducer and the second region associating with a region of the ultrasound analysis object distal from the ultrasound transducer. The invention further laterally controls the two-dimensional brightness of the ultrasound image between the first region and the second region by controlling the amplification of the ultrasound signal using a first gain control circuit for controlling the signal amplification of the first region and a second gain control for controlling the signal amplification of the second region.

By allowing lateral gain control, the user has the ability to control more carefully the image on the ultrasound display. Therefore, variations caused by local gas bubbles or other object features can be viewed more precisely.

A further technical advantage of the present invention is that of providing lateral gain control in ultrasound analysis methods and systems beyond the known areas of cardiac analysis. The simplicity of the present invention makes its use practical in a wide variety of applications beyond cardiac analysis.

The present invention permits the system operator to vary laterally the gain of the received signal as a function of the position of the beam. The invention provides, moreover, at least one operator adjustable LGC command for varying system gain as a function of the displacement of a particular scan line from a reference point, the gain along the scan line as a function of its distance from the system's transducer being substantially constant.

Other technical advantages are readily apparent to one skilled in the art from the following figures, description, and claims.

For a more complete understanding of the present invention and advantages thereof, reference is now made to the following description which is to be taken in conjunction with the accompanying drawings in which like reference numbers indicate like features and wherein:

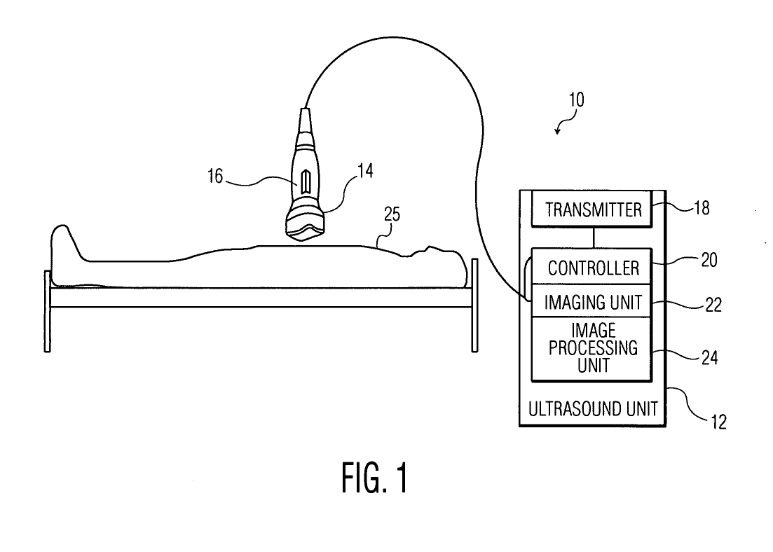

FIG. 1 is a diagram illustrating the use of an ultrasound diagnostic system that may use the present invention;

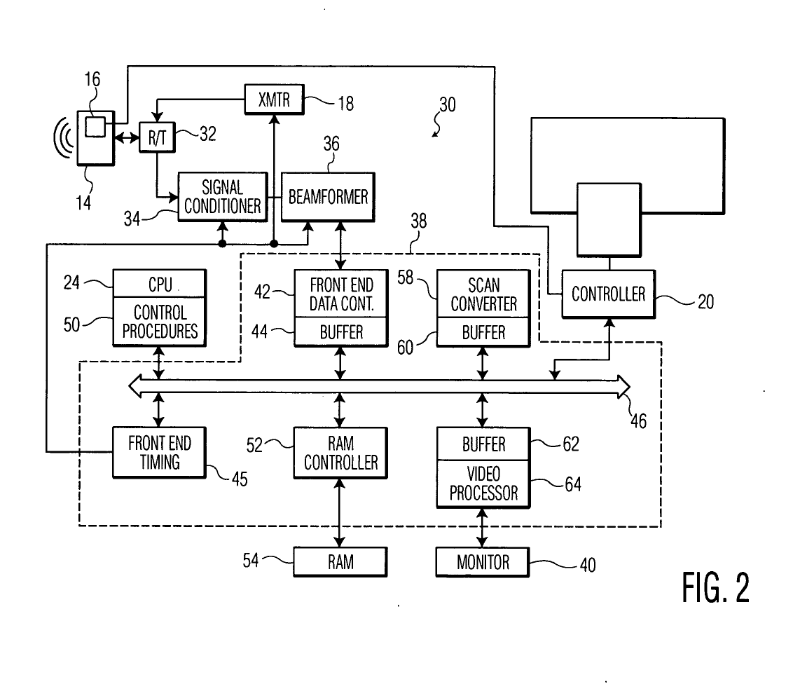

FIG. 2 is a block diagram of an ultrasound system in accordance with the preferred embodiment of the present invention;

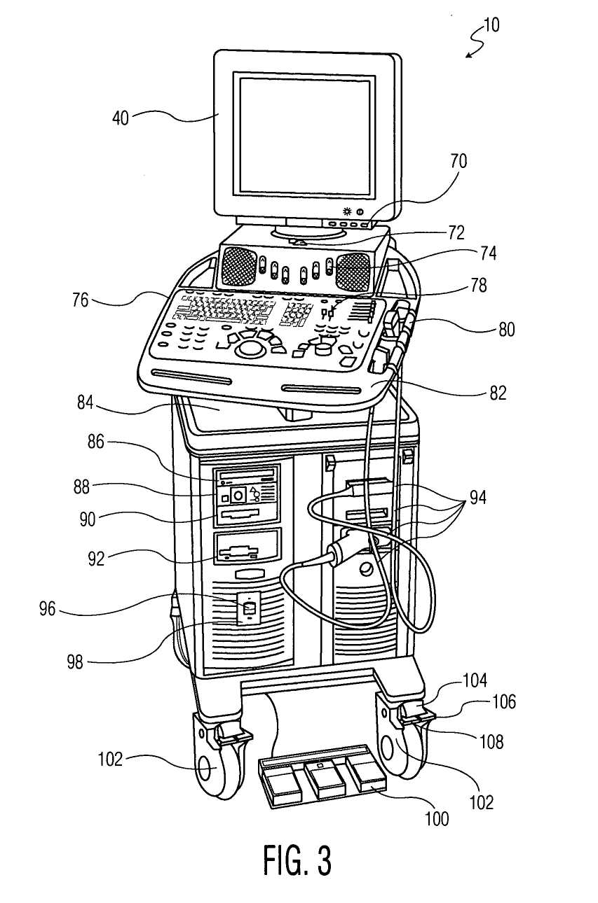

FIG. 3 shows an ultrasound imaging system that may employ the lateral gain control method and system of the present invention;

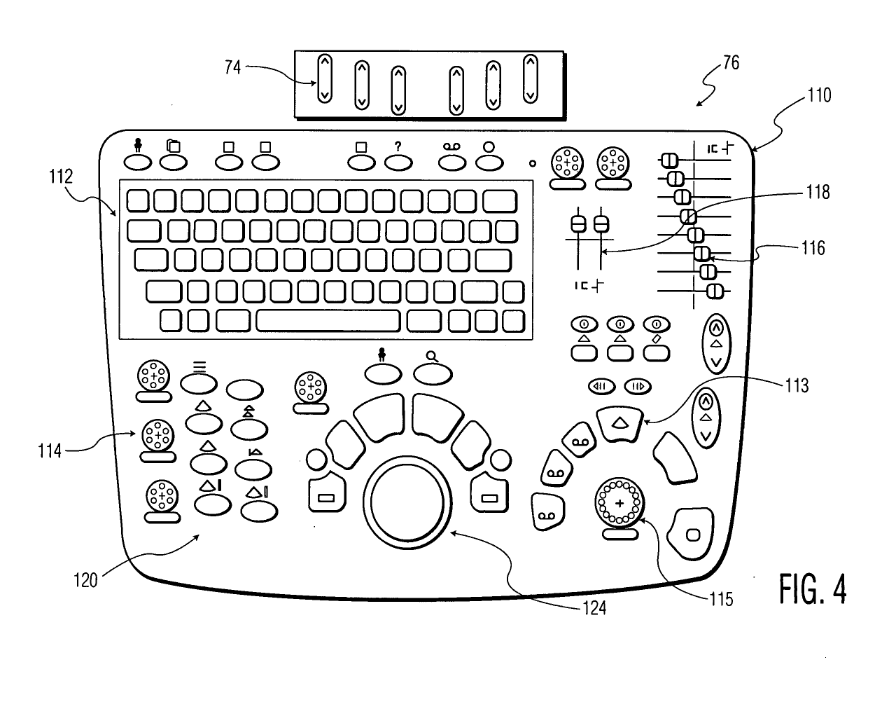

FIG. 4 portrays in more detail the control panel of the ultrasound imaging system of the present invention including the lateral gain controls of the present invention;

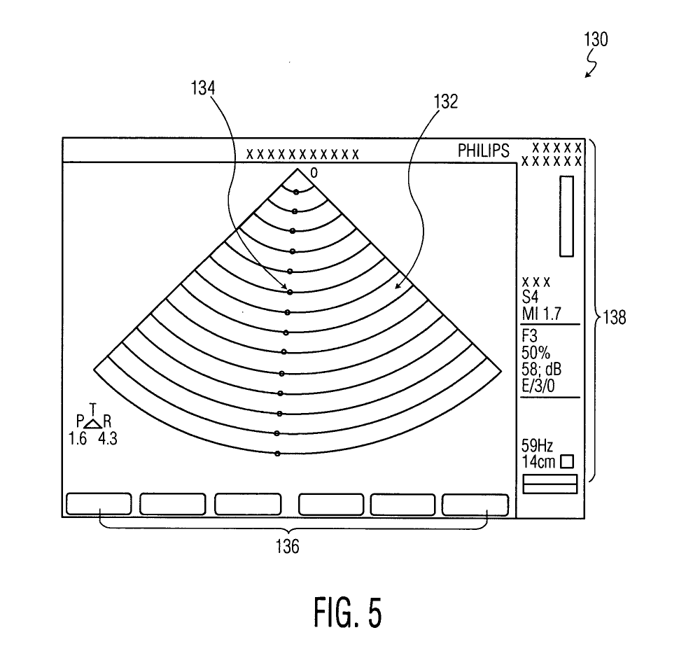

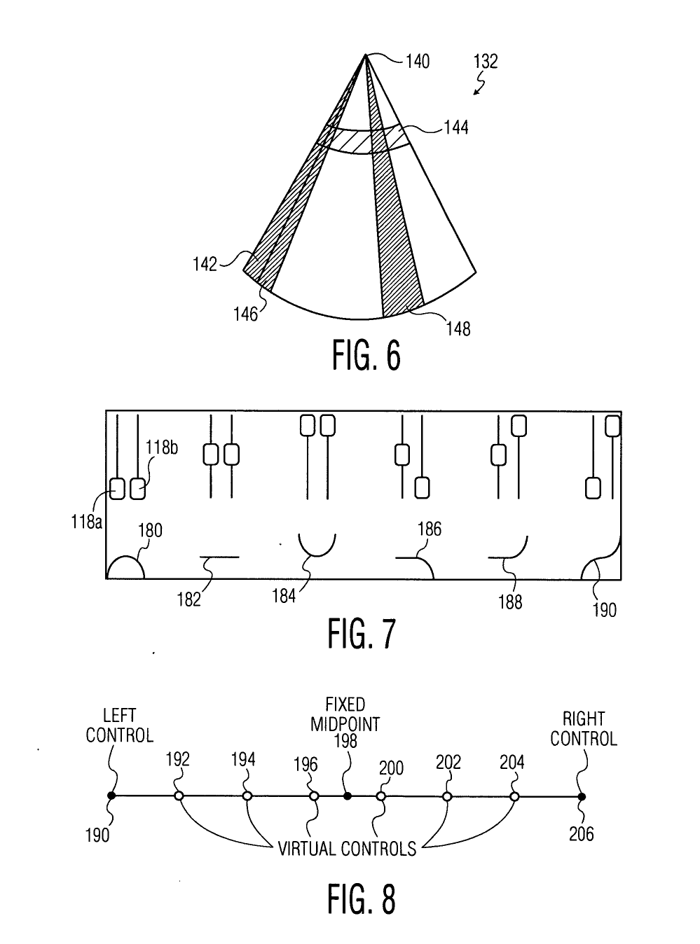

FIGS. 5 and 6 provide views of an ultrasound image including the lateral gain control display of the present invention;

FIG. 7 depicts the variable positions that the sliding lateral gain control of an embodiment of the present invention;

FIG. 8 shows diagrammatically the control response from the variable positioning the sliding lateral gain controls of FIG. 7;

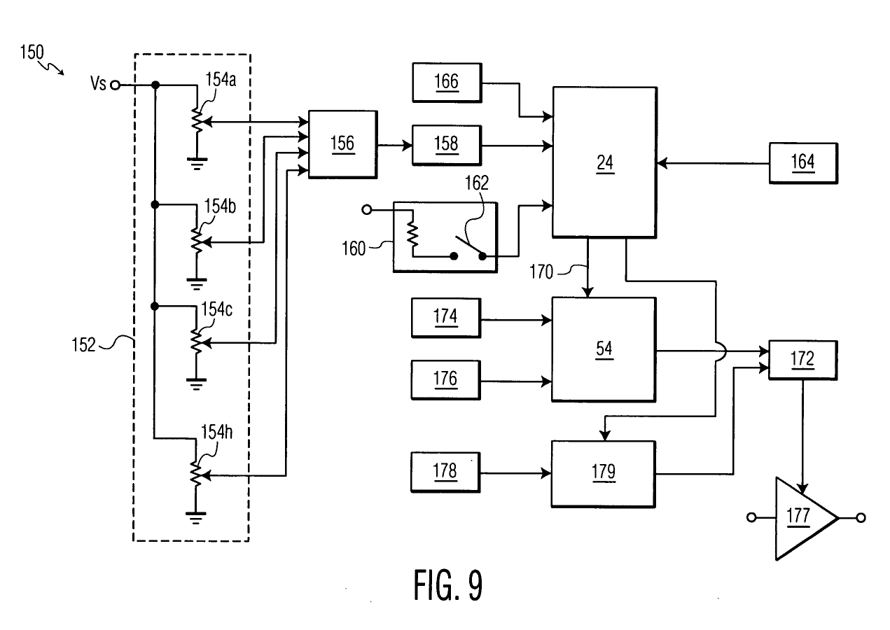

FIG. 9 depicts a lateral gain control circuit that the present invention may employ; and

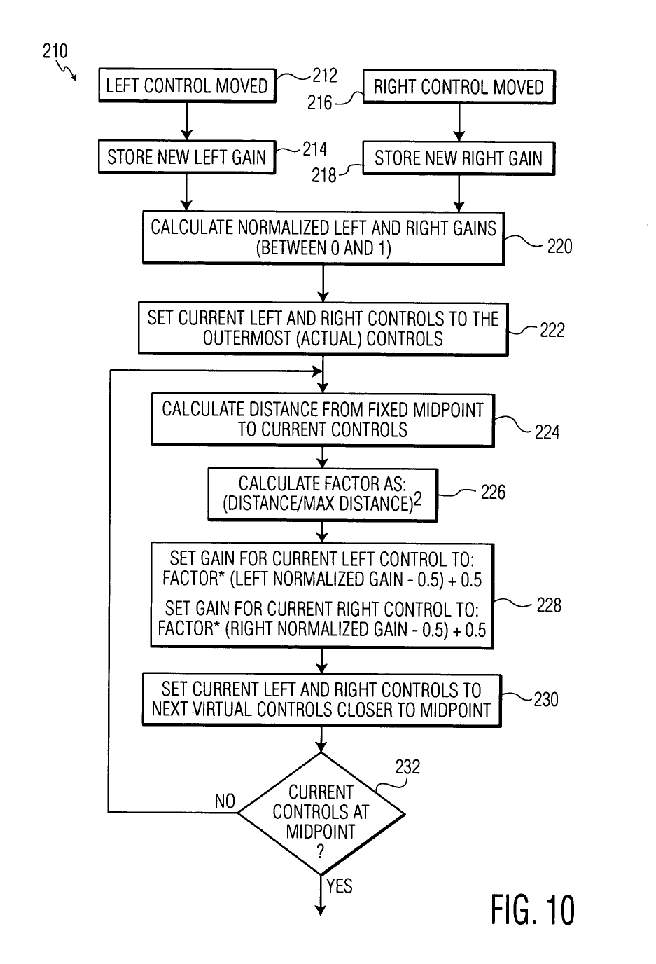

FIG. 10 provides a flow diagram for the general use lateral gain control process and circuitry of the present invention.

The preferred embodiment of the present invention and its advantages are best understood by referring to FIGS. 1 through 10 of the drawings, like numerals being used for like and corresponding parts of the various drawings.

FIG. 1 shows a simplified block diagram of an ultrasound imaging system 10 that may use the concepts presented in the preferred embodiment of the present invention. It will be appreciated by those of ordinary skill in the relevant arts that ultrasound imaging system 10, as illustrated in FIG. 1, and the operation thereof as described hereinafter is intended to be generally representative of such systems and that any particular system may differ significantly from that shown in FIG. 1, particularly in the details of construction and operation of such system. As such, ultrasound imaging system 10 is to be regarded as illustrative, exemplary, and not limiting as regards the invention described herein or the claims attached hereto.

In certain circumstances, when it is desirable that a piece of hardware possess certain characteristics, these characteristics are described more fully in the following text. The required structures for a variety of these machines may appear in the description given below. Machines that may be modified in accordance with the teachings of the present invention include those manufactured by such companies as AGILENT TECHNOLOGIES, PHILIPS MEDICAL SYSTEMS INTERNATIONAL, GE MEDICAL SYSTEMS, and SIEMANS MEDICAL SYSTEMS, as well as other manufacturers of ultrasound equipment.

Ultrasound imaging system 10 generally includes ultrasound unit 12 and connected transducer 14. Transducer 14 includes spatial locator receiver (or simply “receiver”) 16. Ultrasound unit 12 has integrated therein spatial locator transmitter (or simply “transmitter”) 18 and associated controller 20. Controller 20 provides overall control of the system by providing timing and control functions. As will be discussed in detail below, the control routines include a variety of routines that modify the operation of receiver 16 so as to produce an ultrasound image as a live real-time image, a previously recorded image, or a paused or frozen image for viewing and analysis. The differing functions and uses of the ultrasound imaging system that may employ the present invention are described more fully below.

Ultrasound unit 12 is also provided with imaging unit 22 for controlling the transmission and receipt of ultrasound, and image processing unit 24 for producing a display on a monitor (See FIG. 3). Image processing unit 24 contains routines for rendering an ultrasound image which may include, for example, a three-dimensional ultrasound image. Transmitter 18 should be located in an upper portion of ultrasound unit 12 so as to obtain a clear transmission to receiver 16. As modern ultrasound units are often configured in a cart format, such mounting should not be a problem, such that the details thereof are omitted.

During freehand imaging, a user moves transducer 14 over subject 25 in a controlled motion. Ultrasound unit 12 combines image data produced by imaging unit 22 with location data produced by the controller 20 to produce a matrix of data suitable for rendering onto a monitor. Ultrasound imaging system 10 integrates image rendering processes with image processing functions using general purpose processors and PC-like architectures. On the other hand, use of ASICs to perform the stitching and rendering is possible.

FIG. 2 is a block diagram 30 of an ultrasound system in accordance with the preferred embodiment of the present invention. The ultrasound imaging system shown in FIG. 2 is configured for the use of pulse generator circuits, but could be equally configured for arbitrary waveform operation. Ultrasound imaging system 10 uses a centralized architecture suitable for the incorporation of standard personal computer (“PC”) type components and includes transducer 14 which, in a known manner, scans an ultrasound beam, based on a signal from a transmitter 18, through an angle. Backscattered signals, i.e., echoes, are sensed by transducer 14 and fed, through receive/transmit switch 32, to signal conditioner 34 and, in turn, to beamformer 36. Transducer 14 includes elements, preferably configured as a steerable two-dimensional array. Signal conditioner 34 receives backscattered ultrasound signals and conditions those signals by amplification and forming circuitry prior to their being fed to beamformer 36. Within beamformer 36, ultrasound signals are converted to digital values and are configured into “lines” of digital data values in accordance with amplitudes of the backscattered signals from points along an azimuth of the ultrasound beam.

Beamformer 36 feeds digital values to application specific integrated circuit (ASIC) 38 which incorporates the principal processing modules required to convert digital values into a form more conducive to video display that feeds to monitor 40. Front end data controller 42 receives lines of digital data values from beamformer 36 and buffers each line, as received, in an area of buffer 44. After accumulating a line of digital data values, front end data controller 42 dispatches an interrupt signal, via bus 46, to shared central processing unit (CPU) 48, which may be a MOTOROLA PowerPC. CPU 48 executes control procedures 50 including, for example, procedures that are operative to enable individual, asynchronous operation of each of the processing modules within ASIC 38. More particularly, upon receiving an interrupt signal, CPU 48 feeds a line of digital data values residing in buffer 42 to random access memory (RAM) controller 52 for storage in random access memory (RAM) 54 which constitutes a unified, shared memory. RAM 54 also stores instructions and data for CPU 48 including lines of digital data values and data being transferred between individual modules in ASIC 38, all under control of RAM controller 52.

Transducer 14, as mentioned above, incorporates receiver 16 that operates in connection with transmitter 28 to generate location information. The location information is supplied to (or created by) controller 20 which outputs location data in a known manner. Location data is stored (under the control of the CPU 48) in RAM 54 in conjunction with the storage of the digital data value.

Control procedures 50 control front end timing controller 45 to output timing signals to transmitter 18, signal conditioner 34, beamformer 36, and controller 20 so as to synchronize their operations with the operations of modules within ASIC 38. Front end timing controller 45 further issues timing signals which control the operation of the bus 46 and various other functions within the ASIC 38.

As aforesaid, control procedures 50 configure CPU 48 to enable front end data controller 44 to move the lines of digital data values and location information into RAM controller 52 where they are then stored in RAM 54. Since CPU 48 controls the transfer of lines of digital data values, it senses when an entire image frame has been stored in RAM 54. At this point, CPU 48 is configured by control procedures 50 and recognizes that data is available for operation by scan converter 58. At this point, therefore, CPU 48 notifies scan converter 58 that it can access the frame of data from RAM 54 for processing.

To access the data in RAM 54 (via RAM controller 52), scan converter 58 interrupts CPU 48 to request a line of the data frame from RAM 54. Such data is then transferred to buffer 60 of scan converter 58 and is transformed into data that is based on an X-Y coordinate system. When this data is coupled with the location data from controller 20, a matrix of data in an X-Y-Z coordinate system results. A four-(4-) dimensional matrix may be used for 4-D (X-Y-Z-time) data. This process is repeated for subsequent digital data values of the image frame from RAM 54. The resulting processed data is returned, via RAM controller 52, into RAM 54 as display data. The display data is stored separately from the data produced by beamformer 36. CPU 48 and control procedures 50, via the interrupt procedure described above, sense the completion of the operation of scan converter 58. Video processor 64, such as the MITSUBISHI VOLUMEPRO series of cards, interrupts CPU 48 which responds by feeding lines of video data from RAM 54 into buffer 62, which is associated with the video processor 64. Video processor 64 uses video data to render an ultrasound image on monitor 40.

One such system that embodies the techniques of the present invention is the EnVisor Series Ultrasound System manufactured by Philips Medical Systems. The EnVisor series is a powerful ultrasound imaging and image review tool capable of imaging in a variety of modes, including a three-dimensional mode and a panoramic imaging mode. Storing, managing, and reviewing images, as well as performing measurements and calculations using a comprehensive analysis package is possible using a system such as the EnVisor Ultrasound System. Intended uses of such a system include a variety of diagnostic ultrasound applications. These are outlined in the following Table 1.

TABLE 1
Ultrasound System Lateral Gain Control Uses

| | | | PW | CW | | Color | |
|---|---|---|---|---|---|---|---|
| Imaging | 2D | M- | Dop- | Dop- | Color | Power | Calcs |
| Application | Mode | Mode | pler | pler | Flow | Anglo | Analysis |
| Abdominal | X | | X | | X | X | X |
| Cardiac | X | X | X | X | X | X | X |
| Gyneco- | X | | X | | X | X | X |
| logical | | | | | | | |
| Intra- | X | | X | | X | X | X |
| operative | | | | | | | |
| Musculo- | X | | | | | | |
| skeletal | | | | | | | |
| Neonatal | X | | X | | X | X | X |
| Head | | | | | | | |
| Obstetrical | X | X | X | | X | X | X |
| Pediatric | X | X | X | X | X | X | X |
| Small Parts | X | | X | | X | X | X |
| Transcranial | X | | X | | X | X | X |
| Trans- | X | X | X | X | X | | X |
| esophageal | | | | | | | |
| Vascular | X | | X | | X | X | X |

Thus, the ultrasound imaging system in which the lateral gain control method and system of the present invention may be employed may provide two-dimensional analysis, M-mode analysis, pulsed wave analysis, continuous wave analysis, and support color flow studies. Moreover, color power angio and calcs analysis may be achieved with a system that employs the teachings of the present invention. With such a system, abdominal studies may be performed with fundamental or tissue harmonic imaging to obtain images that can be used for detecting abdominal organ abnormalities, evaluating organ size and texture, determining size, contour, and potency of vessels, characterizing obstructions, determining blood flow patterns and velocities, and guiding a biopsy needle, for example, may all be improved with the lateral gain control provided by the present invention. Cardiac studies may be performed with fundamental or tissue harmonic imaging to obtain images that can be used for detecting abnormalities in heart anatomy and blood flow, determining the blood flow patterns and velocities in the heart and associated, vessels, and imaging and measuring anatomic parameters of the heart and associated vessels.

Gynecological studies are performed with fundamental or harmonic tissue imaging to obtain images that can be used for visualizing female reproductive organs, determining blood flow patterns and velocities, guiding a biopsy needle, and detecting structural abnormalities.

Intraoperative studies are performed during surgery to obtain images that can be used to help the surgeon locate and visualize anatomical structures, visualize blood flow patterns and quantify velocities, and image and measure anatomical and physiological parameters of interest. Musculoskeletal studies may be performed to obtain images that can be used for evaluating tendon, ligament, and muscle size and contour, detecting pathology and other abnormalities, and evaluating integrity of tendons and ligaments.

Neonatal head studies are performed to obtain an image of brain structures that can be used to detect abnormalities, such as abnormal ventricle size or a shift in the midline or flow abnormalities. In addition, neonatal head studies are often performed to detect bleeding. Moreover, obstetrical studies may be performed with fundamental or harmonic tissue imaging to obtain images of the fetus that can be used for detecting maternal or fetal structural abnormalities, imaging and measuring anatomic parameters of the fetus, and determining blood flow patterns and velocities. Vascular studies may be performed to obtain images that can be used for detecting vessel size, contour, characterizing obstructions and abnormalities, determining blood flow patterns and velocities, imaging and measuring anatomic parameters of vessels, and detecting structural irregularities.

FIG. 3 shows ultrasound imaging system 10 for using the LGC features of the present invention. As FIG. 3 shows, monitor 40 is controlled by monitor controls 70 and may be positioned using rotation lock lever 72. Monitor 40 presents ultrasound images and related data to the user. Soft keys 74 are programmable according to the particular function which ultrasound imaging system 10 performs such as those which have been already been described. System control panel 76, which is described in more detail below, contains variable controls, including lateral gain controls 78, as well as transducer holder 80 for holding transducer 14 and handles 82 for permitting easy movement of ultrasound imaging system 10. Height release handle 84 permits adjustable positions system control panel 76 and monitor controls 70 according to the user's height.

Ultrasound imaging system 10 further includes drive 86 and physic panel 88, as well as optical disk drive 50 and floppy disk drive 52 for receiving recordable media. Transfer conductors 94 supply data and power to transducer 14. On/off button 96 and reset button 98 control power to ultrasound imaging system 10. Using foot switch 100, a user can control operation of ultrasound unit 12 through foot control. Movement of ultrasound imaging system 10 occurs through the use of wheels 102, which wheel release 104, wheel lock 106 and swivel lock 108 control.

Foot switch 100 allows three of the most commonly used functions to be operated with the user's foot. This keeps the users hands free to manipulate the transducer and the other system controls. The footswitch has three pedals that are configurable during 2D. Footswitch 100 behaves differently while the stress function is active.

FIG. 4 shows system control panel 76 providing horizontal surface 110 that contains keyboard 112 a user may press, rotary controls 114 a user may turn, and TGC 116 slide controls that move left and right. The two LGC slide controls 118 of the present invention move up and down. Notice that keyboard 112, rotary controls 114, and slide controls 116 and 118 are grouped along with other controls with related functions. For example, Doppler controls 120 are grouped in the lower left corner.

Soft keys 74 are oval keys above system control panel 76 and below monitor 40. The functions of the soft keys 74 change depending on the mode, the application, the preset, and the transducer. The function of each soft key 74 is described above the soft key on the bottom of the imaging screen. To use a soft key 74, a user presses the up or the down arrow on the key to choose or change the selection that corresponds to the key on the imaging screen.

Trackball 124, enter key 126, and select key 128 work together somewhat like a computer mouse. Moving trackball 124 is like moving the mouse. Pressing enter key 126 is like pressing the left button on a mouse. In reviewing an image, pressing select key 128 is like pressing the right button on a mouse. To click an item on monitor 40 or to choose an option, the user moves trackball 124 to place the cursor over the item or the option and then presses enter key 126.

FIG. 5 shows imaging screen 130 that monitor 40 may display. Imaging screen includes scan sector 132, along which lateral gain line 134 sweeps. Also appearing on screen 130 are soft key displays 136, which display the functions to be performed by soft keys 74 of control panel 76. Imaging screen 130 also includes settings and patient display region 138. While scan sector 132 may vary, such as when multiple scan sectors 132 are displayed, instead of a single scan sector, soft keys functions 136, settings and patient information 138, however, always remain in the same location.

The various components of scan sector 132 appear diagrammatically at FIG. 6, which illustrates the scan format and some of the terminology to be used in the discussion of the present invention. Scan sector 132 is typically in the form of a wedge having a point of origin 140. Scan lines, such as 142, represent a transmitted and received signal at a particular angle, as measured with respect to a reference, such as one extremity of the scan region. These scan lines generally are not directly displayed, but are processed through the well-known technique of scan conversion, which forms no part of the instant invention, and will not be discussed herein. Region 144 is an example of TGC gain modification and may be varied by an operator in a band as a function of its radial distance from origin 140. Region 146 is an example of gain modification in accordance with the principles of the present invention. An operator may vary the gain in subsector 146, which may be a region having a predetermined angular size. Lateral gain control is constant as a function of its radial distance from origin 140. It may also be constant within the subsector 146. Subsector 148 is another example of where lateral gain may be used. It should be appreciated that the entire scan sector 132 may be subdivided into a predetermined number of subsectors, not illustrated, with each subsector having an associated LGC function.

The real time functions of ultrasound imaging system 10 and their associated controls can be classified as imaging modes, applications, and other functions. Imaging modes functions that change (or potentially change) the screen format. Applications functions that do not change the screen format, but take control of system resources, and possibly display dialog boxes, to accomplish a given task.

The LGC functions of the present invention have application in a wide variety of imaging modes, a few of which are here described. For example, the lateral gain control of the present invention may be used with gray scale real time two-dimensional imaging. LGC may also have application in the Color Flow mode, which provides in real time, two-dimensional imaging showing color overlaying a gray scale image in which the color is determined from the amount and direction of moving blood. The Color Angio mode of operation, which provides in real time two-dimensional imaging showing color overlaying a gray scale image in which the color is determined from the amplitude of moving blood. Moreover, the MMode, which is a gray scale time motion display having a selectable scroll rate may use the present invention. Other operational modes which may make use of the lateral gain control of the present invention include the Color MMode, which is a time motion display showing color overlaying gray scale in which the color is determined from the amount and direction of moving blood. Finally, the Doppler preview function, which presents a real time two-dimensional imaging (gray scale with optional color flow overlay) allowing prepositioning of the Doppler cursor, may also use the simplified lateral gain control of the present invention.

Once an imaging modality has been chosen, there are additional modes, controls, and system features that can be used to enhance, format, or annotate the image display. This is the point at which the LGC functions of the present invention come into play. Lateral gain controls are controls that give the user the ability to adjust the relative brightness laterally across an ultrasound image. These controls have not previously been used on specialty or general imaging ultrasound systems. Lateral gain controls on specialty ultrasound systems allow users to balance laterally image brightness in non-cardiac applications which would contribute to overall image quality. The LGCs work in conjunction with overall gain, TGCs and power to adjust the image brightness. In addition, LGCs enable certain measurements such as fetal head diameter to be taken in situations where they could not otherwise be reliably taken due to too much variability in brightness across the image. Although LGC sliders 118 are slider switches, other possible embodiments include paddle switches, rocker switches, and knobs.

As for the present embodiment, LGC sliders 118 adjust the amplification of returning ultrasound signals within the left and right halves of the image area. Referring to FIG. 7, moving an LGC slide potentiometer 118a up increase the amplification within its respective half of the image, while moving an LGC slide potentiometer 118a down will decrease the amplification within its respective half of the image. The left LGC slide potentiometer 118a controls the amplification on the side of the image nearest the transducer dot. The right LGC slide potentiometer 118b controls the amplification on the side of the image farthest from the transducer dot. For each control, the amplification increases with the distance from the center line of the image.

The algorithm for calculating LGC profile from two controls allows two continuously-variable gain adjustment controls to generate an entire LGC gain profile normally requiring many more controls. The profiles 180 through 190 of FIG. 7 are limited to compound parabolic curves comprised of left hand half-parabolas joined at a fixed midpoint to independent right hand half-parabolas, which half-parabolas may take on different positive, zero, or negative curve angles. The left and right gain controls 118a and 118b govern the gain at the left and right ends of the compound curve respectively. The gain set by each control may range both below and above the fixed midpoint gain. Some examples of control positions and the resulting gain profiles are shown in FIG. 7 as profiles 180 through 190.

To calculate the gain profile, a larger number of virtual gain controls are assigned along the distance of the profile. The gain profile is then represented by the gain value at each virtual gain control. The concept of virtual gain controls is illustrated in FIG. 8. In particular, the various points of control values that may exist from left control point 190, through midpoint control 198, and on to right control point 206 include the other virtual control points 192, 194, 196, 200, 202, and 204. These points more of less correspond to the various positions of left gain control 118a and right gain control 118b. Calculation of a new gain profile is started when either the left or the right gain control is moved. This calculation is implemented through an LGC circuit, such as that of FIG. 9, to follow the logic described in FIG. 10. Note that the normalized gain of the midpoint is fixed at 0.5.

Although a variety of circuit configurations may achieve the LGC functions of the present invention, one such LGC circuit 150 appears in FIG. 9. In FIG. 9, box 152 represents a user interface, by which an operator may select LGC gain values for each subsector where LGC is desired. The use of operator adjustable potentiometers 154a through 154h is shown, however, with the present invention either a set of such potentiometers or other electrical configurations may be employed to achieve the various control setpoints of FIGS. 7 and 8. These potentiometers may be of the linear, as opposed to a rotary type, such as the potentiometers used to control the TGC gain profile on such systems. The analog control voltages generated by elements 154a through 154h are converted to digital format by analog multiplexer 156 and analog-to-digital converter 158. This digital signal is applied to CPU 24. While CPU 24 may be a dedicated processor, in the preferred embodiment, this processing function is implemented through the use of multiple processors, which are also used to control other system functions, such as beam formation. Also supplied to CPU 24 is an LGC enable command 160, which may take the form of off/on switch 162, or otherwise implemented in software instructions. An additional input to CPU 24 is the output of TGC generator 164, which provides a TGC identification and value, and which may be implemented using structure similar to that used to generate the LGC commands. Also supplied to CPU 24 is the output of transducer identification generator 166 which provides information regarding the type of transducer being used.

When the LGC controls are enabled, CPU 24 will generate an LGC gain value for each scan line, which corresponds to the control signals selected by an operator via LGC control 118. The LGC profile may be generated by retrieving precalculated values entered in internal memory of CPU 24 or it may be calculated as needed. FIG. 8, below illustrates in more detail how the present invention makes of use of only two LGC slider controls 118 to achieve the differing LGC control effects.

The LGC profile data is read into line gain RAM 54 via Data Line 170. Although it does not form part of the present invention, in the preferred embodiment, Line 170 also contains gain information related to the earlier mentioned compensation for transducer gain variations. This transducer gain compensation is stored in memory and retrieved in response to a command (not shown) related to the transducer type or its operating frequency. Thus, for a given scan line, the data stored in RAM 54 represents the sum of the LGC signal, and the probe compensation signal. Data is retrieved from RAM 54 and applied to adder 172 in response to information regarding the particular scan line being generated, which is provided by line counter 174, and the line type, (e.g., Doppler, Color Flow, 2-D Imaging) as indicated by line type counter 176.

In the preferred embodiment, also applied to adder 172 is information related to the TGC gain profile. Data is retrieved from RAM 54 in response to depth (as measured with respect to origin 140 of FIG. 6) information provided by depth counter 178.

The combined output signal of adder 172 is supplied via data line 180 to variable gain amplifier 68. Depending on the nature of amplifier 68, the signal contained on line 65 may remain in digital form, or if an analog control signal is required, Adder 64 may include a digital-to-analog converter. Any change to the settings of potentiometers 154a-154h will result in new data being generated by processor 52, and supplied via data line 53 to RAM 54, which in turn updates the data stored in 54. If the LGC control 48 is not enabled, then the data contained in data line 53 will only relate to the transducers probe configuration.

The present invention may also implement a gain save function for storing the current system gain settings as the system acquires each view for subsequently automatically restoring them. With this feature, the first time the ultrasound system acquires a view, the current gain settings (as defined by the current system control settings) are adjusted to control the way the image display appears to the user. These settings are stored with the view when the view is acquired. Each subsequent time the view is selected for acquisition, the gain settings that were stored the previous time the view was acquired are used. If the user manipulates the gain settings, then the new settings are stored with the view when the view is acquired. If the gain settings are not changed, then the original settings are preserved.

FIG. 10 provides flow chart 210 for describing the process of the present invention for using a dual LGC control switch. Beginning at step 212, the process senses the movement of left LGC control 118a (FIG. 7) and stores, at step 214, the new left gain value. Then, if right LGC control 118b is sensed to have moved, at step 216, the process stores the new right gain value at step 218. Next, at step 220, the process calculates normalized left and right gain values to have a range between 0 and 1. At step 222, the process sets the current left and right controls to the outermost or actual controls. Then, at step 224, the distance from a fixed midpoint to the current control position is calculated. At step 226, a factor is calculated as the square of the distance measured relative to the maximum distance for the sensed signals. Step 228 sets the gain for the current left and right controls to equal the calculated factor times the left normalized value minus 0.5 (i.e., normalized left[or right] gain minus 0.5). The value 0.5 is then subtracted from the resulting product. At step 230, the process sets the current left and right controls to the next virtual controls closer to the midpoint. Then, at query 232, the test occurs of whether the current controls are at the midpoint. If so, then the value is used for the LGC signal that a circuit such as that of FIG. 9 uses to control the lateral gain of the ultrasound image display.

In summary, the present invention provides a method and system for laterally controlling the gain of an ultrasound image for use in a general ultrasound imaging system. The method includes steps of generating an ultrasound image of an ultrasound analysis object using an ultrasound sensing circuit for sensing an ultrasound signal returning from an ultrasound analysis object to an ultrasound transducer. The ultrasound image includes a first region and a second region. The first region and the second region together form the ultrasound image, with the first region associating with a region of the ultrasound analysis object proximate to said ultrasound transducer. The second region associates with a region of said ultrasound analysis object distal from said ultrasound transducer. The method further includes the steps of laterally controlling in two-dimensions the brightness of the ultrasound image between the first region and the second region by controlling the amplification of the ultrasound signal using a first gain control circuit for controlling the signal amplification of the first region and a second gain control for controlling the signal amplification of the second region.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A method for laterally controlling the gain of an ultrasound image for use in a general ultrasound imaging system, the method comprising the steps of:
generating an ultrasound image of an ultrasound analysis object using an ultrasound sensing circuit for sensing an ultrasound signal returning from an ultrasound analysis object to an ultrasound transducer, said ultrasound image comprising a first region and a second region, said first region and said second region together forming completely said ultrasound image with said first region associating with a region of said ultrasound analysis object proximate to said ultrasound transducer and said second region associating with a region of said ultrasound analysis object distal from said ultrasound transducer;
laterally controlling in two dimensions the brightness of said ultrasound image between said first region and said second region by controlling the amplification of said ultrasound signal using a first gain control circuit for controlling the signal amplification of said first region and a second gain control for controlling the signal amplification of said second region.

2. The method of claim 1, further comprising the step of laterally controlling in two dimensions the brightness of said ultrasound image using a first slider switch for controlling the signal amplification of said ultrasound signal using said first region and a second slider switch for controlling the signal amplification of said second region.

3. The method of claim 1, further comprising the step of laterally controlling in two dimensions the brightness of said ultrasound image using a first rocker switch for controlling the signal amplification of said ultrasound signal using said first region and a second rocker switch for controlling the signal amplification of said second region.

4. The method of claim 1, further comprising the step of generating said ultrasound image of an ultrasound analysis object, wherein said ultrasound analysis comprises a gynecological analysis object.

5. The method of claim 1, further comprising the step of generating said ultrasound image of an ultrasound analysis object, wherein said ultrasound analysis comprises an abdominal analysis object.

6. The method of claim 1, further comprising the step of generating said ultrasound image of an ultrasound analysis object using a pulsed wave ultrasound signal.

7. The method of claim 1, further comprising the step of generating said ultrasound image of an ultrasound analysis object using a continuous wave ultrasound signal.

8. A general ultrasound imaging system providing lateral gain control for laterally controlling the gain of an ultrasound image, the system comprising:
ultrasound image forming circuitry for generating an ultrasound image of an ultrasound analysis object using an ultrasound sensing circuit for sensing an ultrasound signal returning from an ultrasound analysis object to an ultrasound transducer, said ultrasound image comprising a first region and a second region, said first region and said second region together forming said ultrasound image with said first region associating with a region of said ultrasound analysis object proximate to said ultrasound transducer and said second region associating with a region of said ultrasound analysis object distal from said ultrasound transducer; and
a lateral gain control circuit for laterally controlling in two-dimensions the brightness of said ultrasound image between said first region and said second region by controlling the amplification of said ultrasound signal using a first gain control circuit for controlling the signal amplification of said first region and a second gain control for controlling the signal amplification of said second region.

9. The system of claim 8, further comprising a first slider switch for controlling the signal amplification of said ultrasound signal using said first region and a second slider switch for controlling the signal amplification of said second region.

10. The system of claim 8, further comprising a first rocker switch for controlling the signal amplification of said ultrasound signal using said first region and a second rocker switch for controlling the signal amplification of said second region.

11. The system of claim 8, further comprising a first paddle switch for controlling the signal amplification of said ultrasound signal using said first region and a second paddle switch for controlling the signal amplification of said second region.

12. The system of claim 8, further comprising the step of generating said ultrasound image of an ultrasound analysis object using a pulsed wave ultrasound signal.

13. The system of claim 8, further comprising the step of generating said ultrasound image of an ultrasound analysis object using a continuous wave ultrasound signal.

14. The system of claim 8, further comprising the step of generating said ultrasound image of an ultrasound analysis object using an M-mode ultrasound signal.

15. A storage medium for storing instructions for use in a general ultrasound imaging system, said instructions for providing lateral gain control for laterally controlling the gain of an ultrasound image, the storage medium comprising:
ultrasound image forming instructions for generating an ultrasound image of an ultrasound analysis object using an ultrasound sensing circuit for sensing an ultrasound signal returning from an ultrasound analysis object to an ultrasound transducer, said ultrasound image comprising a first region and a second region, said first region and said second region together forming said ultrasound image with said first region associating with a region of said ultrasound analysis object proximate to said ultrasound transducer and said second region associating with a region of said ultrasound analysis object distal from said ultrasound transducer; and
lateral gain control instructions for laterally controlling in two-dimensions the brightness of said ultrasound image between said first region and said second region by controlling the amplification of said ultrasound signal using a first gain control circuit for controlling the signal amplification of said first region and a second gain control for controlling the signal amplification of said second region.

16. The storage medium of claim 15, further comprising instructions for laterally controlling in two dimensions the brightness of said ultrasound image using a first slider switch for controlling the signal amplification of said ultrasound signal using said first region and a second slider switch for controlling the signal amplification of said second region.

17. The storage medium of claim 15, further comprising instructions for laterally controlling in two dimensions the brightness of said ultrasound image using a first rocker switch for controlling the signal amplification of said ultrasound signal using said first region and a second rocker switch for controlling the signal amplification of said second region.

18. The storage medium of claim 15, further comprising instructions for generating said ultrasound image of an ultrasound analysis object, wherein said ultrasound analysis comprises a gynecological analysis object.

19. The storage medium of claim 15, further comprising instructions for generating said ultrasound image of an ultrasound analysis object, wherein said ultrasound analysis comprises an abdominal analysis object.

20. The storage medium of claim 15, further comprising instructions for generating said ultrasound image of an ultrasound analysis object using a pulsed wave ultrasound signal.

21. The storage medium of claim 15, further comprising instructions for generating said ultrasound image of an ultrasound analysis object using a continuous wave ultrasound signal.
